(19) United States
(12) Patent Application Publication
(10) Pub. No.: US 2005/0181379 A1
(43) Pub. Date: Aug. 18, 2005

(54) Method and device for isolating and positioning single nucleic acid molecules

(75) Inventors: Xing Su, Cupertino, CA (US); Narayanan Sundararajan, San Francisco, CA (US); Tae-Woong Koo, San Francisco, CA (US)

Correspondence Address: Julia A. Hodge;c/o Blakely, Sokoloff, Taylor & Zafman LLP, 12400 Wilshire Boulevard, Seventh Floor, Los Angeles, CA, 90025, US

(73) Assignee: INTEL CORPORATION, Santa Clara, CA (US)

(21) Appl. No.: 10/781,238

(22) Filed: Feb. 18, 2004

(51) Int. Cl. C12Q 1/68; G01N 33/53; C12P 19/34

(52) U.S. Cl. 435/6; 435/7.5; 435/91.2

(57) ABSTRACT

The disclosed methods and devices provides a method and device for isolating and positioning a single polymer molecule, such as a nucleic acid strand, for sequencing, and a method for manufacturing such a device. The method and device can be used for sequencing individual polymer molecules, such as ribonucleic acid (RNA) or deoxyribonucleic acid (DNA).

### TECHNICAL FIELD

Methods and devices for molecular detection, immobilization, isolation, positioning and reactivity are disclosed.

### BACKGROUND

The sensitive and accurate detection, isolation, and identification of single molecules from biological and other samples has widespread application in medical diagnostics, pathology, toxicology, environmental sampling, chemical analysis, forensics and numerous other fields. To date, however, dependable methods of single molecule detection have proven to be an elusive goal. One problem in being able to detect and isolate a small object such as a single molecule is that as the object to be detected gets smaller, it becomes harder to distinguish from the medium surrounding it. In instances where fluorescent molecular labels have been used to aid detection in solution, the single fluorescent molecule must be distinguishable from the background associated with the solution. For single molecule detection, the smallest possible sample volumes are used because the signal from a single molecule is independent of the sample volume. However, the background is always proportional to the sample volume and therefore single molecule detections are based upon the use of sample volumes of 10 pL or less in order to minimize the background contribution.

Because of this small volume limitation, methods for isolating and positioning single molecules, such as fluorescently labeled DNA fragments, for further analysis, have relied on methods such as hydrodynamic focusing to attain sample volumes of about 1 to about 10 pL. In hydrodynamic focusing, a sample stream is introduced into a rapidly flowing sheath stream from a small orifice. The focused sample stream is then crossed with a tightly focused excitation laser beam which is focused to a diameter of from about 10 μm to less than 1 μm. The emitted light is collected by imaging detection optics such as a high numerical aperture microscope objective, passed through a spatial filter or slit and imaged onto a sensitive detector.

The methods relying on hydrodynamic focusing have resulted in only limited success, with only about a 50% probability of detecting a single molecule in a one second time scale (see Ambrose et al. Chem. Rev. 99: 2929-2956 (1999)). Because of this limitation, this method has not been successful in isolating and sequencing single polymer molecules, such as the nucleic acid molecules DNA and RNA.

### BRIEF DESCRIPTION OF THE DRAWINGS

In order that the disclosed methods and devices may be better understood, several embodiments thereof will now be described by way of example only and with reference to the accompanying drawings in which,

FIGS. 1A and 1B depict single molecule supports in accordance with this disclosure;

FIGS. 2A-2D depict the immobilization of a single polymer molecule on a support surface such as on a slide, a fiber optic tip or a microchannel in accordance with this disclosure;

FIG. 3 are digital photographs of streptavidin-coated beads attached to single DNA molecules that are immobilized within microchannels in accordance with this disclosure;

FIG. 4 depicts how a molecular carrier device interacts with a microfluidic single molecule polymer sequencing system in accordance with this disclosure. Please note that the figures are not to scale.

### DETAILED DESCRIPTION OF THE PRESENTLY PREFERRED EMBODIMENTS

These above-described problems have been solved by methods and devices disclosed herein for positioning a single molecule in a given area without the use of expensive tools. The disclosed methods and devices allow for a molecule to be transported to a specific sub-micron area with greater than 99% efficiency in about a millisecond to about a microsecond time scale.

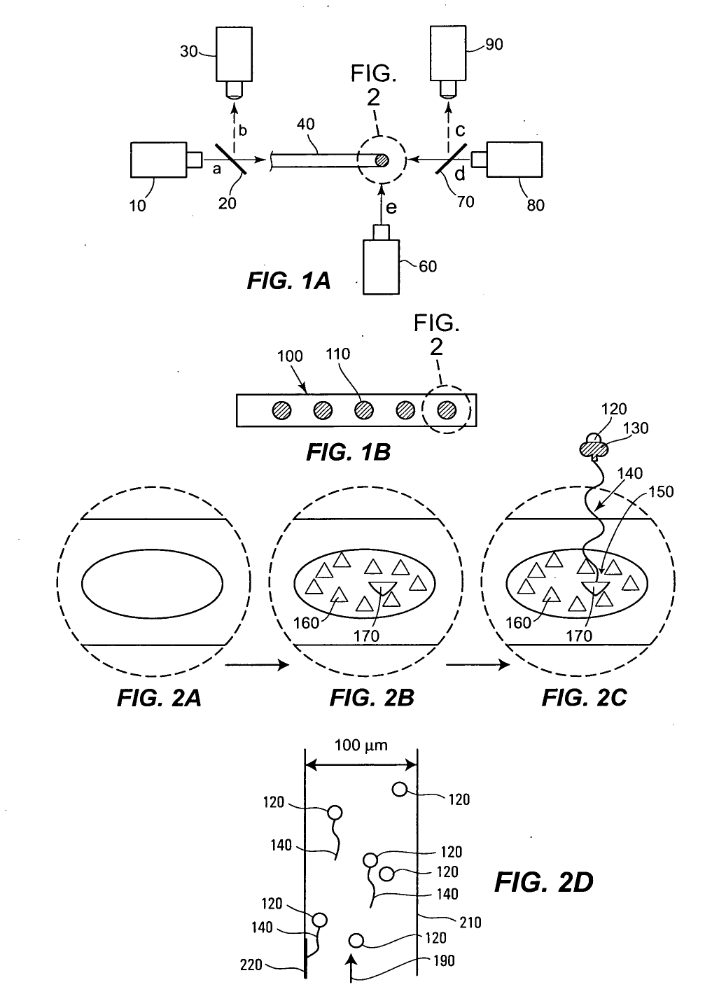

Referring to FIGS. 1A-1B and 2A-2D, polymer molecules 140 capable of being modified include a covalent molecular arrangement of monomers. Examples of such polymer molecules include, but are not limited to, nucleic acids such as DNA and RNA, proteins, carbohydrates and other oligosaccharides, plastics, resins, and the like. For ease of illustration, nucleic acids will be used to exemplify the disclosed methods and devices; however, the disclosed methods and devices are not limited to this example.

Modifications 130 and 150 to the polymer molecule 140, as shown in FIG. 2C, may include any chemical functional group interchange as well as standard molecular labeling techniques. The particular type of modification is chosen to maximize its binding potential with the specific binding molecule and minimize its potential for binding to the functional non-binding molecule or the surface of the support material used in the disclosed methods and devices. Examples of such modifications include, but are not limited to, small functional group changes, such as thiol-modified polymers, amino-modified polymers, aldehyde-modified polymers, carboxy-modified polymers, and the like. Polymers can also be modified with labels or tags that are commonly used in the art. For nucleic acids such labels include, but are not limited to, biotin, fluorescein, digoxigenin, and the like. Such modifications are well known in the art and commercial nucleic acid synthesis vendors provide such modification services (e.g. Qiagen-operon, Valencia, Calif.).

A linear polymer to be immobilized can be modified with either the same (“symmetric modification”) or different (“asymmetric modification”) chemical modifications at each of its two ends. For example a particular polymer molecule can be modified with a thiol group at both ends or with a thiol group at one end and a biotin group on the other end. Asymmetric modification has the advantage that the polymer molecule can be attached at one end through a particular type of attachment, e.g. a the thiol group/gold interaction, leaving the other end free (“free end”) for other manipulations, e.g. labeling with biotin such that it is available to bind streptavidin, avidin, or a streptavidin or avidin modified substrate.

A “specific binding molecule” or “specific binding agent” 170 (see FIGS. 2B and 2C) as used herein is a molecule that can form a strong interaction with the polymeric modification. For example, gold forms a covalent binding interaction with thiol-modified polymer molecules; antibodies are available which selectively bind such molecular labels as fluorescein and digoxigenin, and avidin and streptavidin have a non-covalent binding interaction with biotin with an energy equivalent to some covalent bonds. Specific binding molecules 170 include chemical modifications of a substrate surface with small functional groups which can specifically bind to the chemical modification on the polymer. For example, aldehyde modified surfaces easily attach to amino group modified polymer molecules. The property of this latter interaction has resulted in a variety of commercially available biotin labeled or tagged molecules that can be used to immobilize a molecule on a solid support which is functionalized with avidin or streptavidin molecules. The term “antibody” as used herein includes polyclonal and monoclonal antibodies as well as fragments thereof, recombinant antibodies, chemically modified antibodies and humanized antibodies, all of which can be single-chain or multiple-chain.

A “functional non-binding molecule” or a “functional non-binding agent” 160 (see FIGS. 2B and 2C) as used herein is a molecule which does not form a strong interaction with the polymeric modification. For example, platinum (Pt), and copper (Cu) do not have a binding interaction with thiol groups; a carboxy-modified substrate will not bind to thiol modified polymers; bovine serum albumin (BSA) and bovine IgG (BIgG) do not have a binding interaction with biotin; and streptavidin or avidin do not bind to digoxigenin.

In one embodiment, the specific binding molecule 170 and the functional non-binding molecule 160 used are approximately the same size and molecular weight. For example, Au (MW 197) is of a similar size and molecular weight as Pt (195), but not Ag (MW 107.9) or Cu (65.5). Likewise, BSA (MW 65 kD) is of a similar size and molecular weight as avidin (MW 66 kD).

The microarea 110 (FIG. 1B) can be of any particular size. In one embodiment, at least one of the dimensional distances (e.g. diameter, height, width, etc.) of the micro area 110 should be at least two times the length of the polymer molecule to prevent the polymer molecule 140 from attaching at both modified termini. For example, for a DNA molecule comprising about 50,000 base pairs, this distance is about 17 microns long. Therefore, when such a DNA molecule is used this microarea 110 can range from about 17 microns to about 70 millimeters.

In one embodiment of the disclosed methods and devices, the specific binding molecule 170 is mixed with an effective molar amount of the functional non-binding molecule 160 such than only one modified polymer molecule 130-150 can be immobilized in a given microarea 110 on a solid support 40 or 100 (FIGS. 1A and 1B). The molar ratio of specific binding molecule 170 to functional non-binding molecule 160 (the “substrate ratio”) can be changed and experimentally verified depending on the desired distance between the molecules to be immobilized. Any substrate ratio can be used. Ratios of the specific binding molecule to the functional non-binding molecule (the “substrate ratio”) may range from about 1:10¹⁰ to about 10:1 depending on the particular combination of specific binding molecule and functional non-binding molecule. For example, if gold is the specific binding molecule and copper is the functional non-binding molecule, then a ratio of about 1:10⁸ respectively is advantageous. If monomeric avidin is the specific binding molecule and BSA is the functional non-binding molecule, then a ratio of about 1:10⁷ is advantageous.

The molar ratio of modified polymer to specific binding molecule 170 to functional non-binding molecule 160 (the “target ratio”) can also be changed and experimentally verified depending on the desired distance between the molecules to be immobilized. Any target ratio can be used. Target ratios may range from about 1:10¹⁰ to about 1:0 depending on the particular combination of specific binding molecule and modified polymer. For example, if monomeric avidin is the specific binding molecule and the polymer is streptavidin, then a ratio ranging from about 1:10 to about 1:1000 is advantageous.

In one embodiment, one may use a formulation containing only specific binding molecule and no functional non-binding molecule. In this embodiment if a symmetrically modified polymer is used, most of the polymer molecules will be attached to the substrate at both ends (no “free end” for further modification) and only a few polymer molecules will be immobilized with a free end. Because polymer molecules with free ends are limited in this embodiment they will have a lower density, however they are still easily detected and isolated. Polymer molecules with no free ends do not interfere with the isolation of polymer molecules with free ends.

In one embodiment, the specific binding molecule 170 used may have multiple binding sites. For example, normal avidin and streptavidin have about 4 binding sites in each molecule. In this embodiment a suitable amount of a blocking molecule may be added such that there is only one effective binding site per specific binding molecule. For example if avidin is used, free biotin can be mixed with the biotin-modified polymer in about a 3:1 ratio such that 3 of the 4 binding sites are blocked from binding the modified polymer. As used herein “effective binding site density” is the density of total binding sites multiplied by the ratio of blocking molecules to target molecules, assuming the total number of blocking molecules and target molecules is far greater than the total number of binding sites.

Various types of solid supports 40, 100 (FIGS. 1A and 1B) can be used in the disclosed methods and devices. Examples of suitable solid supports include, but are not limited to, plates, slides, films, strips, rods, tubes, beads, and the like. These supports can be made from a variety of materials including, but not limited to, metal, glass or other silica-based materials, polymeric resin-based materials, and the like. For ease of illustration, a metal or glass slide 100 and an optical fiber 40, as shown in FIGS. 1A and 1B, will be used to exemplify the disclosed methods and devices, however, the disclosed methods and devices are not limited to these examples.

Still referring to FIGS. 1A-1B and 2A-2C, the specific-binding 170 and functional non-binding molecules 160 are attached to the solid support 40 or 100 by a variety of methods known in the art depending on the support material and the molecules to be used. For example, if the support is metal, and gold and silicon are the specific binding molecule and the functional non-binding molecule, respectively, standard metal annealing methods may be used. If the support material is glass, and avidin and BSA are the specific binding molecule and the functional non-binding molecule, respectively, standard covalent coupling methods may be used.

Standard covalent coupling methods comprise providing a reactive group either to the molecule to be attached to the surface or to the surface itself. Examples of these reactive groups include, but are not limited to, carboxyl, amino, hydroxyl, hydrazide, amide, chloromethyl, aldehyde, epoxy, tosyl, thiol, and the like, which are commonly used in the art.

For example, aldehyde modified glass surfaces have been shown to especially suitable for the present application for use in proteins for the following reasons. First, the existence of terminal amino groups on the proteins used as functional-nonbinding and functional binding compounds in the disclosed methods and devices ensures their availability for complementary attachment to one or more aldehyde groups on the surface of the support. Second, after reducing the imine produced this group has proven to be very stable over time. Third, the chemistry involved in attaching ligands to either of these groups has been widely explored. Fourth, the reagents involved are readily commercially available.

Aldehyde-modified glass surfaces can be prepared by two processes. The first process involves immersing a polished and NoChromix and Piranha cleaned surface for 30 minutes in a hydrolysed solution of 0.5% glycidyloxypropyltrimethyoxysilane (GPTMS), 4.5% ethyltrimethoxysilane (ETMS) in 50 mM pH 5.7 4-morpholineethanesulfonic acid (MES), followed by a solution of 1 mM sodium periodate (NaIO₄) in pH 7.2 PBS for 1 hr at room temperature (RT). The second process involves sonicating the polished and cleaned surfaces in 2% GPTMS in 95% EtOH/5% deionized water (DI H₂O) for 2 minutes, rinsing with ethanol (EtOH) and drying, and then immersing the surfaces in a solution of 1 mM NaIO₄ in pH 7.2 PBS for 1 hr at room temperature.

The microarea(s) 110 to be coated with the specific binding molecule 170 and non-functional binding molecule 160 mixture may be coated by standard inkjet printing, standard photolithography, contact printing techniques or techniques for microarray fabrication to deposit the specific binding and non-functional binding molecules in given areas on the surface of the support 40, 110. The support 40, 110 can be coated in multiple positions.

In certain embodiments of the disclosed methods and devices, specific areas of the support 40, 110 can be precoated with protecting groups so that these areas cannot be coated with the mixture of the specific binding molecule and the functional non-binding molecule. The specific protecting groups used depend on the type of surface to be protected. Examples of protecting groups for glass substrates include, but are not limited to, substituted and unsubstituted alkyl ethers, substituted and unsubstituted benzyl ethers, silyl ethers, esters, carbonates, sulfonates, and the like. (See e.g., T. Greene ed. “Protecting Groups in Organic Synthesis” (1991)). Removing these protecting groups, either chemically, or mechanically by cleaving or etching the support surface, then exposes a fresh substrate surface which can be coated with the mixture of the specific binding molecule 170 and the functional non-binding molecule 160.

The modified polymer molecule shown at 130, 140, 150 in FIG. 2C can then be immobilized on coated microareas 110 of the support 40 or 100 by contacting the modified polymer molecule with the coated solid support. For example if the substrate is coated with gold, then the thiol-modified polymer is applied over the gold patch allowing the formation of covalent attachment between gold surface and thiol group. Any unbound polymer molecules can be removed by washing the coated area with a buffer solution.

In other embodiments the polymer can be synthesized on the substrate. Using nucleic acids as an example, a polydeoxyadenosine (poly (dA)) primer modified with a thiol group on one end can be first immobilized on a surface using the above methods. Then a template DNA molecule with a polydeoxythymidine (poly (dT)) sequence (either labeled or unlabeled) is allowed to hybridize or anneal to the preimmobilized poly(dA) through adenosine-thymidine hybridization. The poly (dA) sequence can then be extended by a DNA polymerase in the presence of nucleotides and other required reagents. Unused primer molecules can then be separated from the desired, immobilized nucleic acid molecule.

The detection of a single bound polymer molecule 140 and the verification of the spacing between individual bound polymer molecules can be accomplished by a variety of methods depending on the modification at the free terminus of the polymer molecule 130. These methods include, but are not limited to, labeling the immobilized polymer molecule by contacting it with a fluorescently labeled specific binding molecule or other label 120 that is specific for the modification on the polymer's free terminus. For example, if a nucleic acid molecule is modified with biotin at its free terminus, the immobilized nucleic acid can be labeled with avidin-tagged or labeled with fluorescent molecules or with a streptavidin bead. Alternatively the polymer molecule can be detected by contacting the immobilized polymer with a fluorescent dye, label, or stain and detecting the individual polymer molecules and scanning the support for fluorescent emission from the label using a single-photon counting device or some other optical detecting device. Likewise, the nucleic acid molecule can be stained by a nucleic acid specific dye, such as, ethidium bromide.

Detection Unit

The embodiments of the disclosed methods and devices are not limited by the type or arrangement of detection unit used, and any known detection unit may be used in the disclosed methods and device. If the labels are fluorescent, standard light sources 10, 60, or 80, such as those shown in FIG. 1A, can be used to provide the desired absorption wavelength of common fluorescent dye molecules. Examples of such light sources include, but are not limited to, lasers, mercury or xenon gas lamps (Oriel Instruments) and filters (Omega Optical or Chroma). For example, the tip of an optical fiber 40 is used as the support, such light a can be delivered to the molecule through the optical fiber to which the molecule is attached. In such an embodiment, part of the emitted fluorescent light b is captured by the same optical fiber, and travels back to the other end of the optical fiber. A dichroic mirror 20 can be used as part of this detection method to separate beams or waves of excitation light and emitted fluorescence light, by reflecting the back-scattered fluorescent light toward a detector 30. If the fluorescence from the optical fiber interferes with the fluorescence from the attached molecule, or if a collinear geometry is difficult to implement due to the alignment or the size of the instrument, a forward or side scattering geometry c can be used. In a forward-scattering geometry, excitation light d is delivered to the molecule and part of the emitted fluorescent light b is captured by the optical fiber and travels to detector 30 either directly or reflected by dichroic mirror 20. In a side-scattering geometry, excitation light e is delivered to the molecule and part of the emitted fluorescent light b is captured by the optical fiber and travels to detector 3 either directly or reflected by dichroic mirror 20.

Still referring to FIG. 1A, the optical detector 30 or 90 can be any standard optical detector or array of detectors including, but not limited to, photodiode detectors, avalanche photodiode detectors, Charge-Coupled Devices (CCD) arrays of detectors, Complementary Metal-Oxide Semiconductor (CMOS) arrays, intensified CCD cameras, or any other optical detector with reasonable sensitivity and speed.

CMOS arrays using both N-type and P-type transistors may also be used to realize logic functions. CMOS technology has advantages in that little to no static power dissipation when compared to Negative-Channel Metal-Oxide Semiconductor (NMOS) or bipolar circuitry. Power is only dissipated in case the circuit actually switches. This allows integration of many more CMOS gates on an integrated circuit than in NMOS or bipolar technology, resulting in much better performance.

In order to further reduce the fluorescence generated by the optical fiber, the excitation beam can impinge the attached molecule at an angle outside the collection angle of the optical fiber.

Typically about 4% of the impinging light is reflected from the surface, which is considered as a loss in transmission. In another embodiment, the attachment end of the optical fiber can be coated with dielectric materials designed to allow the fluorescence from the attached molecule to enter the optical fiber with low light loss, while reflecting the excitation light and preventing it from entering the optical fiber. A typical dielectric coating can block the excitation light by factor of 106 and transmit more than 96% of the fluorescence light impinging on the coating.

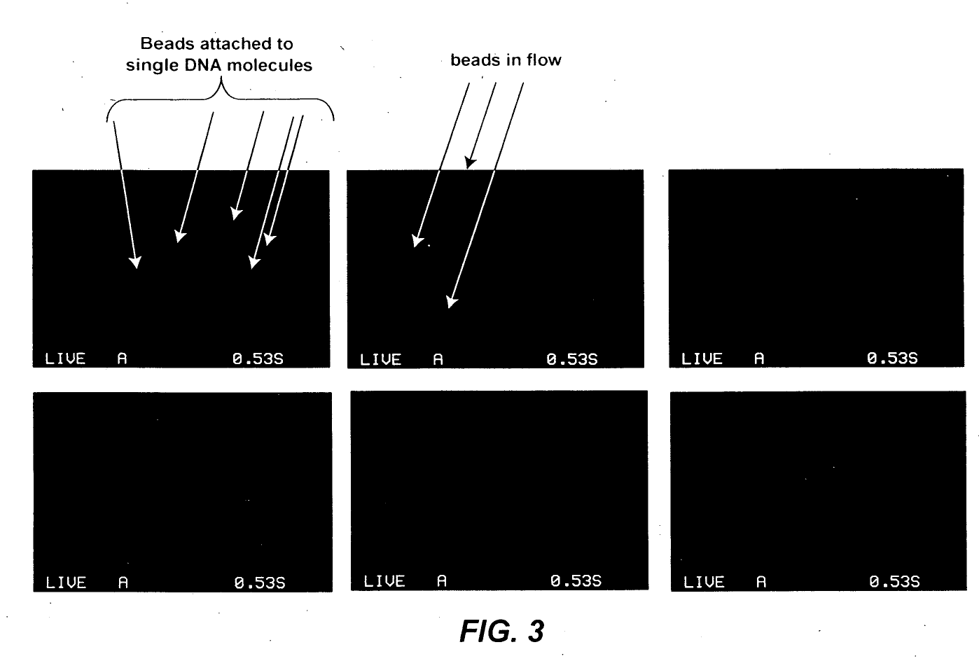

In another embodiment illustrated in FIGS. 2D and 3, the label 120 is a bead and the molecule 140 may detected visually using a microscope or other optically magnifying device. For example, FIG. 3 shows digital photographs of streptavidin-coated beads attached to single DNA molecules that are immobilized within microchannels 210 as also shown in FIG. 2D. In FIG. 2D, beads 120 attached to a single DNA molecule 140 which is also attached to the substrate 220 (large spots) can be differentiated from beads 120 attached to single molecules 140 and unattached beads 120 in the flow 190 After identifying areas where single polymer molecules are attached to the substrate, positions that have single polymer molecules may be marked using microscopy stages and saved for later use.

### EXAMPLE

### Single Molecule Isolation—Sample Preparation for DNA Sequencing

The following is a description of the techniques used to generate the samples shown in the digital photographs of FIG. 3.

Substrate Modification

A glass surface is treated with alkaline solution (NaOH, IN) to expose hydroxyl groups. The hydroxylated surface is subsequently treated with an aldehyde-containing silane reagent (10 millimolar in 95% ethanol) to provide an aldehyde-activated substrate. After washing with ethanol three times, and deionized water three times, the aldehyde-activated substrate is coated with a solution containing avidin and BSA (bovine serum albumin) in certain molar ratio: 1:10 or 1:1000, etc. The aldehydes react readily with primary amines on the proteins to form Schiff's base linkages between the aldehydes and the proteins, i.e., to covalently attach the proteins to the aldehyde-activated substrate surface.

Target Molecule Preparation

A DNA sample is digested with two different restriction enzymes to create DNA fragments having two different ends (e.g., 10 micrograms of yeast DNA is digested in 100 microliters of 1× restriction enzyme digestion buffer (New England Biolabs), containing 50 units of EcoR1 and 50 units of BamH1). About 10 nanograms of a 20 kbp DNA fragment are isolated from agarose gel by methods known by those of ordinary skill in the art. A hairpin-like oligonucleotide (cap-oligo) with a biotin moiety in the middle and a restriction enzyme site at its end is synthesized and ligated to the desired end (determined by the restriction enzyme). After ligation, the DNA has a closed end with a biotin and an open end.

50 microliters of an enzyme solution containing terminal transferase (20 units) and 10 micromolar dATP can be used to add a biotinylated oligonucleotide tail (20-50 nucleotides long) to the open end of the DNA. Other end modification methods can also be used, depending on the final application of the molecule.

Beads for Attachment and Confirmation

Streptavidin coated micro-sphere (fluorescent) of 1 urn can be purchased from a commercial source (Polysciences Inc.)

Microfluidic Chip Fabrication

Designs of the micro fluidic channels to be fabricated were drawn to scale using CAD software. The designs were then printed onto transparencies using a high-resolution printer. The channels were ˜100 μm in width and 2-3 cm in length. “Photoresist on Silicon” masters for micromolding were prepared by standard photolithography using the transparency masks and SU-8 photoresist. These patterned masters were then silanized and used for micromolding with poly (dimethyl siloxane) (PDMS). PDMS precursor was poured onto the silanized master and then cured. The cured PDMS containing the channel structure was then bonded to the modified substrate by applying pressure to enclose the channels.

Single Molecule Isolation

The modified target DNA with biotinylated ends was immobilized on the avidin/BSA substrate within the microfluidic channel by pumping a 10 nM of the target DNA solution through the microfluidic channel for 5 min using vacuum and incubating the solution for an hour. The channel was then washed with 1×PBS 3-5 times to remove any unbound target DNA. Confirmation of the attachment of the target DNA and isolation was performed by flowing a solution of 1 um fluorescent streptavidin-coated polystyrene beads (PS) obtained from Polysciences, Inc. and observing the Brownian motion of the beads attached to the target DNA immobilized on the substrate within the microfluidic channel using fluorescent video microscopy.

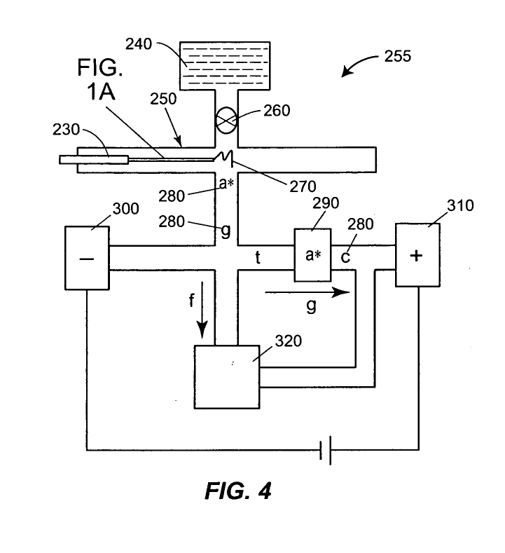

The disclosed methods and devices can be used for sequencing single polymer molecules including nucleic acids such as DNA and RNA. Referring to FIG. 4, in one embodiment, the polymers can be sequenced by placing the molecular carrier in a microfluidic device equipped for single polymer molecule sequencing and detection. The molecular carrier of FIG. 1A is positioned in the system with a positioning device 230 such that a single molecule 270 is positioned in the reaction chamber 250 of the sequencing device 255. The positioning device 230 can be fitted with a seal (not shown) such that the carrier can be moved in and out without causing leakage. Then, using a combination of chemical or enzymatic methods and microfluidics, each monomer (both labeled and non-labeled) from the polymer strand can be sequentially cleaved and transported into a collection volume for detection. For example, if the polymer molecule is a nucleic acid, a buffered enzyme solution 240 with exonuclease activity is then flowed using a flow control device 260 into the reaction chamber 250 of the channel to digest the DNA strand and release the individual labeled or unlabeled nucleotide monomers 280 one at a time. Preferably this enzyme solution is pumped into the reaction chamber 250 at a predetermined rate using the flow control device 260. The cleaved nucleotide monomers 280 are carried/transported in the flow f and g directed through a sample cell 290 where the signal from the monomer or its label is sequentially detected. A electrical field generated by an anode 300 and a cathode 310 may be used to help focus the monomers through the sample cell. The nucleotide monomers may optionally be carried or transported to a collection or waste chamber 320.

The foregoing detailed description of the preferred embodiments of the disclosed methods and devices has been given for clearness of understanding only, and no unnecessary limitations should be understood therefrom, as modifications will be obvious to those skilled in the art. Variations of the disclosed methods and devices as hereinbefore set forth can be made without departing from the scope thereof, and, therefore, only such limitations should be imposed as are indicated by the appended claims.

1. A method for isolating a single polymer molecule comprising:
chemically modifying at least one terminus of a single polymer molecule to form a modified polymer molecule;
coating a microarea on the surface of a solid support with
an amount of a specific binding molecule that binds the modified polymer molecule and,
an amount of a functional non-binding molecule that does not bind with the modified polymer molecule such that the average distance between effective binding sites is two times the polymer's length to form a coated solid support; and
contacting the modified polymer molecule with the coated solid support.

2. The method of claim 1 wherein the polymer molecule is a nucleic acid.

3. The method of claim 2 wherein at least one terminus of the polymer molecule is chemically modified to comprise a thiol, carboxy, or amino group.

4. The method of claim 2 wherein at least one terminus of the polymer molecule is chemically modified with a molecule selected from the group consisting of biotin, digoxigenin, fluorescein, and combinations thereof.

5. The method of claim 2 wherein the specific binding agent comprises gold and the functional non-binding agent comprises silver, copper, magnesium, silicon, gallium or a combination thereof.

6. The method of claim 2 wherein the specific binding agent is avidin or streptavidin and the functional non-binding molecule is a protein.

7. The method of claim 6 wherein the protein is bovine serum albumin.

8. The method of claim 1 wherein the microarea is from about 400 nm² to about 100 mm².

9. The method of claim 1 wherein the average distance between polymer molecules is 1 micron to about 70 mm.

10. The method of claim 1 wherein the solid support is selected from the group of a plate, a slide, a film, a strip, a rod, a tube, and combinations thereof.

11. The method of claim 1 wherein said tube is an optical fiber.

12. The method of claim 1 wherein the surface of the support is precoated with a protecting group.

13. The method of claim 1 further comprising detecting the presence of a single polymer molecule attached to the support.

14. The method of claim 13 further comprising marking the position of a single polymer on the support.

15. A device for isolating or transporting a single polymer molecule comprising:
a solid support comprising a micro area which is coated with an amount of a specific binding molecule admixed with a functional non-binding molecule such that an average distance between effective binding sites is two times a length of the single polymer molecule.

16. A device according to claim 15 further comprising an immobilized polymer molecule.

17. A device according to claim 16 wherein the polymer is a nucleic acid.

18. A device according to claim 15 further comprising a mark of the location of the polymer molecule on the support.

19. The device of claim 15 wherein the specific binding molecule is gold and the functional non-binding molecule is copper, silicon, gallium, or combination thereof.

20. The device of claim 15 wherein the specific binding molecule is avidin or streptavidin and the functional non-binding molecule is bovine serum albumin.

21. The device of claim 15 wherein the microarea is from about 400 nm² to about 100 mm².

22. The device of claim 15 wherein an average distance between polymer molecules is 0.1 microns to about 70 mm.

23. The device of claim 15 wherein the solid support is selected from the group of a plate, a slide, a film, a strip, a rod, and a tube.

24. The device of claim 15 wherein said tube is an optical fiber.

25. The device of claim 15 wherein part of the surface of the support is coated with a protecting group.

26. A method for isolating and sequencing a single polymer molecule comprising:
chemically modifying at least one terminus of the polymer molecule to form a modified polymer molecule;
coating a substrate with
an amount of a specific binding agent that binds the modified polymer molecule and,
an amount of a functional non-binding agent that does not bind with the modified polymer molecule such that the average distance between effective binding sites is two times the polymer's length to form a coated solid support;
adhering the substrate to a microchannel;
flowing the modified polymer molecule into the microchannel;
allowing the modified polymer to adhere to the substrate;
washing the substrate and modified polymer; and
sequencing the modified polymer.

27. The method according to claim 26 wherein the polymer is a nucleic acid.
